(19) United States
(12) Patent Application Publication
(10) Pub. No.: US 2001/0027287 A1
(43) Pub. Date: Oct. 4, 2001

(54) Apparatus for providing coronary retroperfusion and/or left ventricular assist and methods of use

(75) Inventors: Ascher Shmulewitz, Mercer Island, WA (US); John Burton, Minnetonka, MN (US); Robert S. Bley, Menlo Park, CA (US)

Correspondence Address: Robert D. Buyan, STOUT, UXA, BUYAN & MULLINS, LLP, 4 Venture, Suite 300, Irvine, CA, 92618, US

(73) Assignee: Trans Vascular, Inc.

(21) Appl. No.: 09/794,297

(22) Filed: Feb. 27, 2001

Related U.S. Application Data
(63) Continuation of application No. 09/084,513, filed on May 26, 1998, now abandoned.

(51) Int. Cl. A61M 37/00; A61M 5/00

(52) U.S. Cl. 604/7; 604/154

(57) ABSTRACT

Apparatus and methods for reducing the load on a patient's left ventricle while perfusing ischemic myocardium are provided using a first conduit having an inlet end configured for insertion into the left atrium, left ventricle or aorta coupled to a second conduit having an outlet end configured for insertion into the coronary venous vasculature via the coronary ostium. A motor-driven or hydraulically-actuated pump may be coupled between an outlet end of the first conduit and an inlet end of the second conduit to direct flow between the first and second conduits. Control circuitry is provided for use with the motor-driven pump to control the pump with a user selected duty cycle.

### FIELD OF THE INVENTION

The present invention relates generally to treatment and/or diagnosis of ischemic heart disease prior to, during, or after a corrective procedure, such as bypass grafting, heart replacement or angioplasty, and involves perfusing the myocardium with oxygenated blood from the left atrium, left ventricle or aorta using the venous system of the heart.

### BACKGROUND OF THE INVENTION

Each year worldwide several millions of patients undergo cardiac bypass surgery, during which stenosed and atherosclerotic cardiac vessels are replaced with native veins or arteries harvested elsewhere from the body.

A first step in treating or correcting cardiac disease, such as coronary artery disease, is to determine which portions of the heart are most likely to benefit from revascularization. In this manner, the clinician is able to assess the functioning of the myocardium, the location of infarcted or distressed areas, and select an appropriate treatment plan, e.g., an open-chest surgical procedure, so called “keyhole” coronary artery bypass grafting (“CABG”) or angioplasty. Several methods of determining cardiac functioning are described, for example, in Udelson, “Steps Forward in the Assessment of Myocardial Viability in Left Ventricular Dysfunction,” Circulation, 97:833-838 (1998). It would therefore be desirable to provide methods and apparatus that enhance a clinician's ability to better assess left ventricular dysfunction.

Patients often experience irreversible damage to ischemic myocardium while awaiting corrective therapy or surgery. It would therefore be desirable to provide apparatus and methods for percutaneously preserving the myocardium of patients awaiting a corrective procedure.

A number of techniques have been developed to preserve the myocardium during corrective procedures, such as angioplasty and bypass procedures, that involve perfusing the heart using the coronary venous system. For cardiac surgery, a patient's heart is typically stopped, and the patient is placed on a cardiopulmonary bypass machine. Hypothermia is induced and maintained in the heart throughout the bypass operation to reduce necrosis of the myocardium caused by oxygen starvation.

Coronary retroperfusion also may be used as to preserve ischemic myocardium, as described in Kuraoka et al., “Antegrade or Retrograde Blood Cardioplegic Method: Comparison of Post-Surgical Right Ventricular Function and Conduction Disturbances,” Japanese J. Thoracic Surg., 48(5), pp. 383-6, (1995); Ihnken et al., in “Simultaneous Arterial and Coronary Sinus Cardioplegic Perfusion, an Experimental and Clinical Study,” Thoracic and Cardiovascular Surgeon, Vol. 42, pp.141-147 (June 1994); and Lincoff et al., “Percutaneous Support Devices for High Risk or Complicated Coronary Angioplasty,” J. Am. Coll. Cardiol., 17(3), pp. 770-780 (1991)).

Retrograde blood flow through the coronary venous system may be augmented by coronary ostial occlusion, as described in Rudis et al. in “Coronary Sinus Ostial Occlusion During Retrograde Delivery of Cardioplegic Solution Significantly Improves Cardioplegic Distribution and Efficiency,” J. Thoracic & Cardiovasc. Surg., 109(5), pp. 941-946 (1995). In this case, blood flows retrograde to the myocardium and drainage is through the lymphatic system and the Thebesian veins.

Aldea, et al., in “Salvage of Ischemic Myocardium With Simplified and Even Delayed Coronary Sinus Retroperfusion,” Ann. Thorac. Surg., No. 62, pp. 9-15 (1996), describe three techniques for preserving ischemic myocardium during a simulated bypass operation. The first method, referred to as pressure-controlled intermittent coronary sinus retroperfusion (“PICSO”) involves placing a balloon in the coronary sinus, which is periodically inflated and deflated. When the balloon is inflated, blood draining into the coronary sinus is passively redirected in a retrograde manner through the coronary venous system, thereby perfusing the myocardium.

A second method described in the Aldea article is synchronized retroperfusion (“SRP”). In SRP, a balloon is placed in the coronary sinus, and in synchrony with balloon inflation, oxygenated blood is pumped into the coronary sinus so that it flows in a retrograde manner. The balloon is inflated, and blood injected into the coronary sinus, only during diastole. During systole, the balloon is deflated and blood flow into the coronary sinus ceases.

A third method, described in the Aldea article as simplified retroperfusion (“SR”), is similar to SRP, but no balloon is placed in the coronary sinus. Instead, a pump is used to continuously inject blood into the coronary sinus. Apparatus suitable for use with the foregoing methods is described in U.S. Pat. No. 5,597,377 to Aldea.

The foregoing methods generally are used as adjuncts to hypothermia to preserve the myocardium when the heart is stopped for open-heart surgery. “Keyhole” surgical techniques, however, such as developed by Cardio Thoracic Systems, of Menlo Park, Calif., enable coronary artery bypass grafting (“CABG”) to be performed on a beating heart. In accordance with those methods, the heart is not stopped, but instead the bypass surgery is performed while the heart is beating. It therefore would be desirable to provide methods and apparatus that enable the clinician to preserve the myocardium during beating heart cardiac surgery.

In addition, once the bypass operation is completed, the heart is revived and blood flow through the heart is restored to normal. In some cases, however, there may be some difficulty in weaning the patient from the cardiopulmonary bypass machine. In particular, the heart can become overexerted when attempting to restore flow in the arterial system. In these situations, an intra aortic balloon pump (“IABP”) may be used to lower the pressure encountered by the left ventricle during systole.

The intra-aortic balloon pump generally comprises a balloon catheter which is placed in the ascending aorta or aortic arch, and which is cyclically inflated and deflated in synchrony with the heart. In particular, the balloon is inflated during cardiac diastole, so that blood in the aorta is urged into the descending aorta. The balloon is then deflated in anticipation of systole, and reduces the pressure against which the left ventricle ejects blood during contraction.

In “Enhanced Preservation of Acutely Ischemic Myocardium With Transseptal Left Ventricular Assist,” Ann. Thor. Surg. 1994, No. 57, pp. 570-575, Fonger et al., describe an experimental left ventricular assist device (“LVAD”) for use in weaning a cardiac bypass patient from a cardiopulmonary bypass machine. The device comprises a pump having an inlet catheter disposed in the left atrium via a femoral vein and an outlet catheter located in a femoral artery. The article describes that the LVAD device reduces the load on the left ventricle by draining a portion of the blood from the left atrium into the femoral artery.

It also would be desirable to provide apparatus and methods that assist the left ventricle, by reducing the volume of blood pumped by, and thus, the exertion of, the left ventricle in patients awaiting, or who have completed, cardiac bypass surgery.

### SUMMARY OF THE INVENTION

In view of the foregoing, it is an object of the present invention to provide methods and apparatus that enhance a clinician's ability to better assess left ventricular dysfunction by providing coronary retroperfusion.

It is another object of this invention to provide apparatus and methods for preserving ischemic myocardium of patients awaiting corrective procedures.

It also is an object of the present invention to provide methods and apparatus that enable the clinician to preserve the myocardium during beating heart cardiac surgery.

It is a further object of this invention to provide apparatus and methods for providing retrograde short-term perfusion of the myocardium prior to or during a corrective procedure.

It is a further object of this invention to provide apparatus and methods that assist the left ventricle, by reducing the volume of blood pumped by, and thus, the exertion of, the left ventricle in patients awaiting, undergoing, or who have completed, cardiac bypass surgery.

These and other objects of the present invention are achieved by providing apparatus and methods for draining a volume of blood from the left atrium, left ventricle or aorta and directing that blood into the coronary venous vasculature to provide retrograde perfusion of the myocardium.

Apparatus constructed in accordance with the present invention comprises a first conduit having an inlet end configured for insertion into a patient's left atrium, left ventricle or aorta and coupled to a second conduit having an outlet end configured for insertion into the coronary venous vasculature via the coronary ostium. The apparatus may be used for diagnosis of cardiac dysfunction, or prior to, during or after a corrective procedure. A pump, which may be motor driven, hydraulically actuated, or comprise the beating heart itself, is coupled to the circuit formed by the first and second conduits to infuse oxygenated blood into the coronary venous vasculature. Therapeutic agents, such as drugs or bioactive agents, or cooled saline may be added to the blood passing through the circuit.

In accordance with other aspects of the present invention, the coronary ostium may be either partially or fully occluded by the outlet of the second conduit. The pump also may be operated with a duty cycle designed to control a parameter related to the pressure in the coronary venous system, so as to reduce the potential for edema of the venous system. Where the pump is motor-driven, control circuitry optionally may be provided to activate the pump with a user selected duty cycle to reduce exertion of the left ventricle by draining blood from the left atrium or left ventricle and injecting that blood into the coronary venous system to provide retrograde perfusion. A sensor optionally may be coupled in the circuit formed by the first and second conduits to monitor a flow-related parameter.

Methods of implanting and operating apparatus constructed in accordance with the present invention are also provided for post-operative weaning of the patient from cardiac bypass.

### BRIEF DESCRIPTION OF THE DRAWINGS

Further features of the invention, its nature and various advantages will be more apparent from the accompanying drawings and the following detailed description of the invention, in which:

FIG. 1 is a perspective view of a human heart, partly in section, illustrating implantation of a first embodiment of the apparatus of the present invention;

FIG. 2 is a perspective view of the apparatus of FIG. 1;

FIG. 3 is a sectional view of a first conduit constructed in accordance with the present invention;

FIG. 4 is a sectional view of a second conduit constructed in accordance with the present invention;

FIG. 5 is a timing diagram showing an illustrative duty cycle for activation of the pump of the apparatus of FIG. 1;

FIG. 6 is a perspective view of a human heart, partly in section, illustrating implantation of an alternative embodiment of the apparatus of the present invention;

FIG. 7 is a perspective view of the apparatus of FIG. 6;

FIGS. 8A and 8B are, respectively, sectional views of the pump portion of the apparatus of FIGS. 6 and 7 in the outflow and inflow states;

FIGS. 9A and 9B are, respectively, sectional views of an alternative pump portion constructed in accordance with the principles of the present invention;

FIGS. 10A and 10B are, respectively, sectional views of a further alternative pump constructed in accordance with the principles of the present invention;

FIG. 11 is a perspective view of a human heart, partly in section, illustrating implantation of a further alternative embodiment of the apparatus of the present invention; and

FIG. 12 is a side view, partly in section, of a coupler constructed in accordance with the present invention that includes a sensor for monitoring a flow-related or physiologic parameter.

### DETAILED DESCRIPTION OF THE INVENTION

The present invention relates generally to methods and apparatus for diagnosing cardiac dysfunction and for providing short-term (e.g., from a few minutes to several weeks) transvenous myocardial perfusion for patients suffering from ischemic heart disease, such as atherosclerosis, prior to, during or after a corrective procedure, such as cardiac bypass surgery or angioplasty. In accordance with the methods of the present invention, a fluid circuit is formed between the left atrium, left ventricle or aorta and the coronary venous system, so that a volume of the blood in the left atrium, left ventricle or aorta is diverted to the coronary venous system.

In certain embodiments, a motor-driven pump is provided to remove a volume of blood from the left atrium or left ventricle, and is expected to assist the left ventricle by reducing its degree of exertion. The extracted blood is then injected into the coronary venous system to improve perfusion of the myocardium. In other embodiments, an hydraulically-actuated pump may be coupled in the fluid circuit to provide a pressure gradient sufficient to cause flow from the left ventricle to the coronary venous system, or the heart itself may provide the necessary pumping action. Preferably, control circuitry or a mechanical mechanism is provided that limits a pressure-related or flow-related parameter for the flow in the coronary venous system to a value less than a predetermined value.

The fluid circuit of the present invention may be implanted percutaneously, for example, using femoral or jugular/subclavian access sites, directly through small openings in the chest, as in “keyhole” type CABG techniques, or intraoperatively after a thoracotomy. Blood circulated through the fluid circuit may be infused with drugs or bioactive agents, be monitored for flow rate, pressure or other physiologic parameters, or be cooled by an external cooling system or diluted with chilled saline to induce a mild state of hypothermia (e.g., by 2-5° C.).

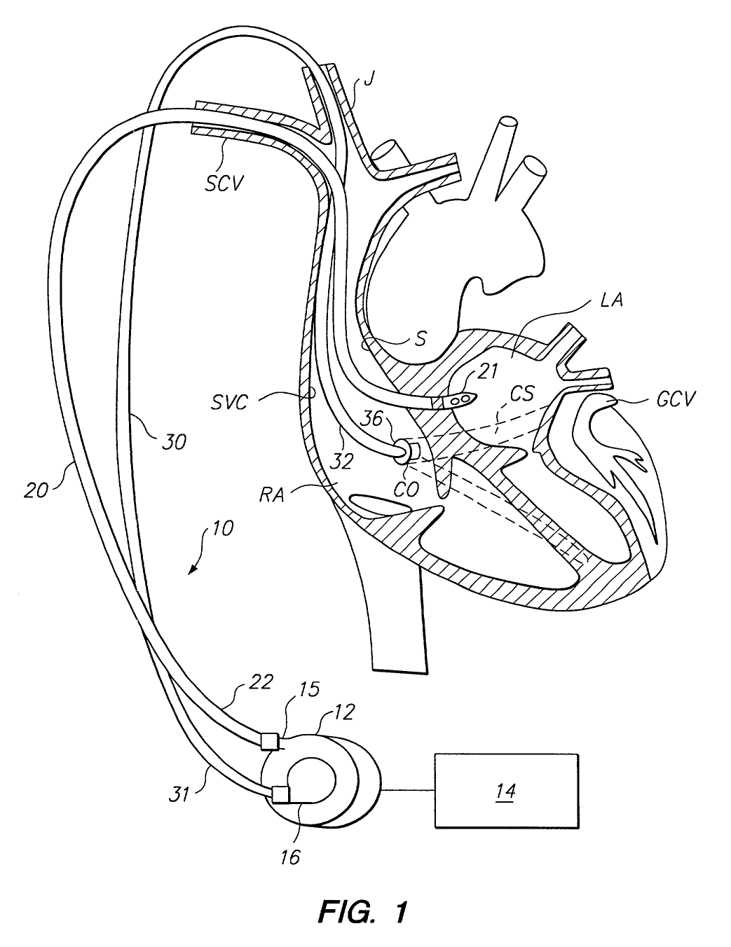

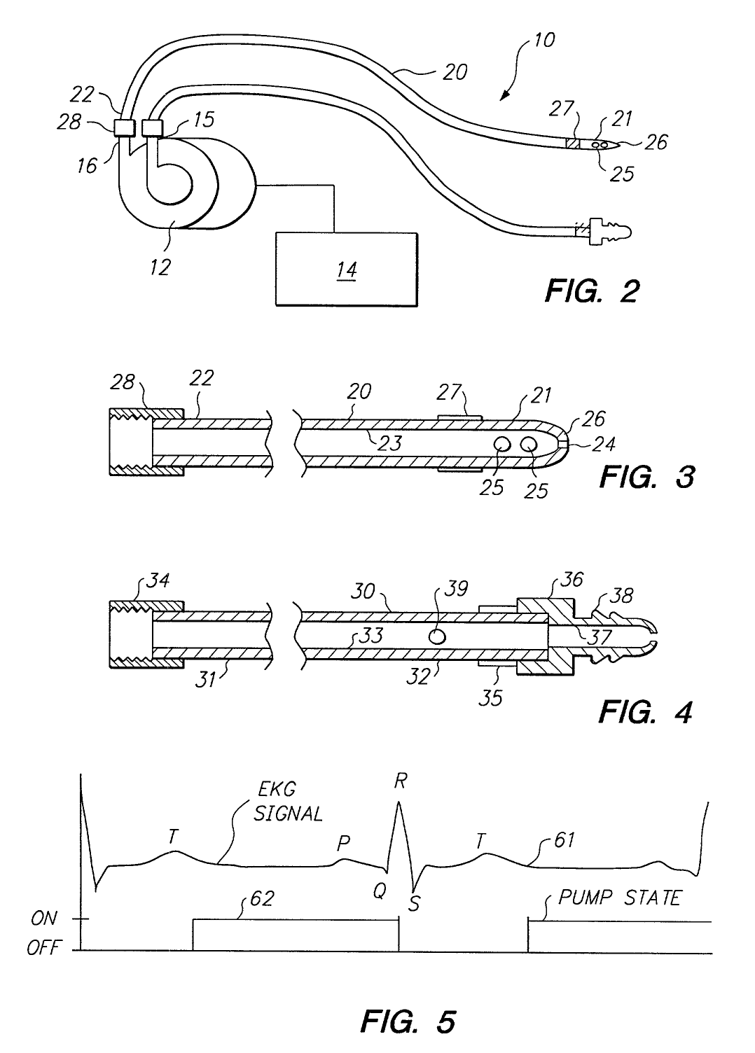

Referring to FIGS. 1 to 4, a first embodiment of apparatus 10 constructed in accordance with the present invention is described. Apparatus 10 comprises conduits 20 and 30 coupled to motor-driven pump 12. Control circuitry 14 controls operation of pump 12 responsive to user selected input. Pump 12 includes inlet port 15 and outlet port 16, and may be constructed in accordance with known techniques used in previously known infusion pumps, such as the Baxter Flow-Gard 6201, Baxter International, Deerfield, Mich., or previously known centrifugal pumps, such as those manufactured by Sarns, Inc., Ann Arbor, Mich.

Conduit 20 has inlet end 21, outlet end 22 and lumen 23 connecting the inlet and outlet ends (see FIG. 3). Inlet end 21 may be transluminally inserted via the right internal jugular vein J (or alternatively, right subclavian vein SCV) and superior vena cava SVC into the right atrium RA, and extends through a puncture in the atrial septum S into the left atrium LA. Inlet end 21 preferably includes central opening 24, plurality of lateral openings 25, and bullet-shaped or conical-shaped tip 26 that enables inlet end 21 to urged along a guide wire (not shown) to penetrate the atrial septum. Inlet end 21 also preferably includes a radio-opaque marker band 27, for example a gold film, that enables the location of the inlet end to be determined using a fluoroscope. Outlet end 22 is coupled to inlet 15 of pump 12 by fitting 28, for example, threads or a quick-connect coupling.

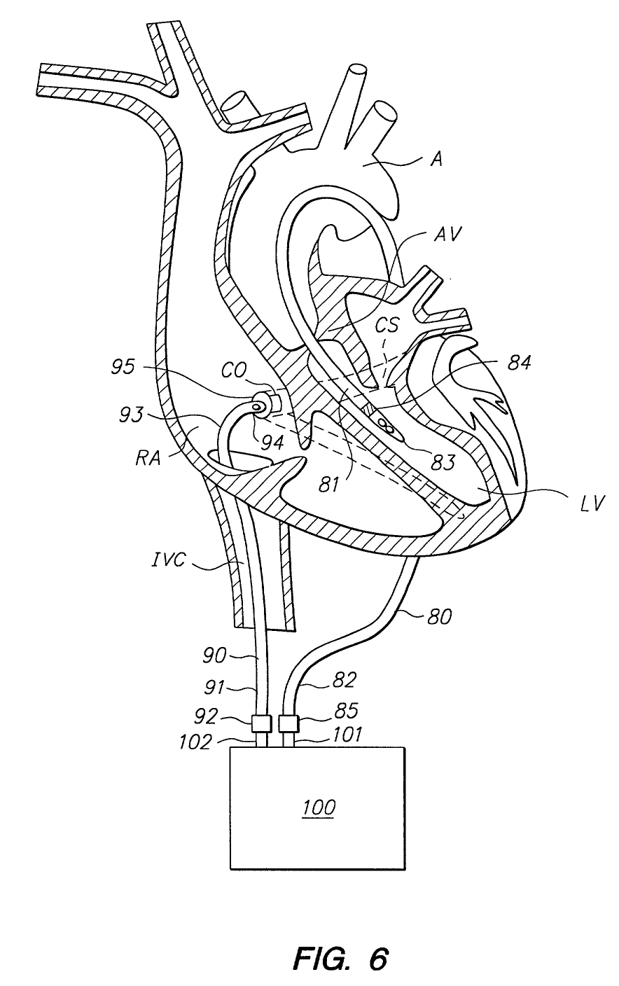

Alternatively, inlet end 21 of conduit 20 may be inserted transluminally and transseptally, as described hereinabove, and then passed through the mitral valve from the left atrium into the left ventricle. It is expected that short-term use of conduit 20 in this manner will not adversely effect the mitral valve. As yet another alternative, described hereinafter with respect to FIG. 6, inlet end 21 of conduit 20 may be inserted transluminally via the femoral artery and aorta into the aortic root, and the passed through the aortic valve into the left ventricle.

Conduit 30 has inlet end 31, outlet end 32 and lumen 33 connecting the inlet and outlet ends (see FIG. 4). Inlet end 31 is coupled to outlet port 16 of pump 12 by fitting 34, which may also be, for example, threads or a quick-connect coupling. Outlet end 32 is transluminally inserted via the right subclavian vein SCV (or right internal jugular vein J) and superior vena cava SVC into the right atrium RA, and extends through the coronary ostium CO into the coronary sinus CS. Outlet end 32 preferably includes radio-opaque marker band 35 and plug 36. Plug 36 has bore 37 and a retention mechanism 38, for example, a plurality of barb or rib-type projections, that engage the interior wall of the coronary sinus to retain the plug in the coronary sinus until forcibly removed. When inserted into the coronary sinus, outlet end 32 may either partially or fully occlude the coronary ostium and permit partial flow from the coronary sinus into the right atrium.

Alternatively, instead of disposing outlet end 32 of conduit 30 in the coronary sinus, outlet end 32 may be advanced through the coronary sinus into another portion of the cardiac venous vasculature, for example, great cardiac vein GCV, to provide more localized retroperfusion of the myocardium. In this case, plug 36 may be configured so that conduit 30 passes through it a predetermined distance, or plug 36 may be omitted entirely. In addition, conduit 30 may include one or more openings 39 for venting a portion of the blood from conduit 30 into the right atrium, for example, when the volume of blood drained from the left atrium or left ventricle to reduce left ventricle exertion is greater than the volume needed to perfuse the venous system.

Conduits 20 and 30 preferably comprise a biocompatible, flexible material typically used in catheters, for example, polyvinyl chloride, polyethylene or silicone. Conduit 30 is preferably more rigid than conduit 20, so that plug 36, if present, may be removably seated in coronary ostium CO by exerting force on inlet end 31 of the conduit. Plug 36 preferably comprises an elastomeric material, such as rubber, latex or silicone.

Control circuitry 14 may be constructed in accordance with previously known designs for circuitry used in controlling infusion pumps, and permits a clinician to input a duty cycle that specifies intervals of activation and deactivation of the pump. Control circuitry 14 cyclically activates and deactivates pump 12 responsive to the input duty cycle. Control circuitry 14 also preferably includes circuitry for measuring the flow rate and pressure of blood flowing through conduit 30, and accordingly may accept as input limit values pressure-related or flow-related parameters, for example, peak pressure, mean pressure, or maximum flow rate. Activation of pump 12 is then controlled so that a measured or computed parameter (based on the measured pressure or flow in conduit 30) does not exceed the limit values.

Thus, for example, control circuitry 14 may accept as an input limit values a value of 60 mm Hg for the peak pressure and a value of 5-100 ml/min for the maximum flow rate attained in conduit 30. Some of the literature suggests that 60 mm Hg is the maximum peak pressure sustainable in the coronary venous system without causing edema of the veins. Control circuitry 14 monitors, via a suitable flow probe disposed on or in conduit 30, the pressure and flow rate in the conduit and shuts off or reduces the speed of pump 12 to maintain the peak pressure and flow rate in the coronary venous system below the input limit values.

Referring still to FIG. 1, implantation of apparatus 10 in accordance with the methods of the present invention is now described. Conduit 20 may be implanted using a transluminal approach that is a variation of the Brockenbrough method of catheterizing the left ventricle. The conventional Brockenbrough technique, which is described in CARDIAC CATHETERIZATION AND ANGIOGRAPHY, W. Grossman, ed., at pages 63-69, published by Lea & Febiger, Philadelphia (1980), employs a catheter and needle combination that is advanced through the right femoral artery and into the right atrium, and used to puncture the septum between the right and left atria.

In accordance with the present invention, a Brockenbrough needle kit, available from United States Catheter and Instrument Corp., Billerica, Mass., is advanced over a guide wire into the right atrium via the right internal jugular vein using a standard Seldinger technique. The Brockenbrough needle is used to puncture the atrial septum, and the transseptal puncture is then dilated using, for example, progressively larger diameter catheters, which are then withdrawn, leaving the guide wire in place.

Next, conduit 20 is slipped over the proximal end of the guide wire, via central opening 24, so that the guide wire passes through lumen 23 and exits through fitting 28. Conduit 20 is then advanced over the guide wire so that inlet end 21 passes through the transseptal puncture and into the left atrium, as determined, for example, by visual confirmation of the location of marker band 27 using a fluoroscope. If desired, the clinician may advance inlet end 21 of conduit 20 through the mitral valve and into the left ventricle. Once inlet end 21 of conduit 20 is positioned in the left atrium or left ventricle, the guide wire is withdrawn proximally through fitting 28. Fitting 28 is then coupled to inlet port 15 of pump 12.

Using standard catheterization techniques, a guide wire is inserted transluminally via right internal jugular vein J (or alternatively, right subclavian vein SCV), through superior vena cava SVC, and into coronary sinus CS via coronary ostium CO. Conduit 30 is slipped over the proximal end of the guide wire, via bore 37 in plug 36, so that the guide wire passes through lumen 33 and exits through fitting 34. Conduit 30 is advanced over the guide wire so that plug 36 passes through coronary ostium CO and becomes lodged in coronary sinus CS. Alternatively, the clinician may advance outlet end 32 of conduit 30 through the coronary sinus and into a selected cardiac vein (e.g., great cardiac vein GCV) under fluoroscopic guidance. Once outlet end 32 of conduit 30 is positioned in the coronary venous vasculature, the guide wire is withdrawn proximally through fitting 34. Fitting 34 is then coupled to outlet port 16 of pump 12, completing implantation of the apparatus.

The clinician then inputs a desired duty cycle and any desired limit values into control circuitry 14 via a suitable input pad or keyboard. Responsive to the duty cycle and limit values input by the clinician, control circuitry 14 cyclically activates pump 12 to drain a desired volume or flow rate of blood from the left atrium or left ventricle through conduit 20, thereby partially unloading the left ventricle. Pump 12 then injects that drained volume of blood into the coronary sinus or selected cardiac vein, thereby providing retrograde perfusion of the myocardium that reduces infarction of the ischemic region of myocardium. It is expected that apparatus 10 will infuse the venous system with blood at flow rates of 5-100 ml/min. Higher rates of drainage from the left atrium or left ventricle may be attained where conduit 30 includes openings 39 (see FIG. 4) for venting a portion of the blood into the right atrium.

Referring now to FIG. 5, an exemplary duty cycle 60 that may be input to control circuitry 14 is described. Waveform 61 of FIG. 5 is that obtained from an electrocardiograph, while waveform 62 corresponds to the on/off state of pump 12. It is contemplated that one mode of operation of pump 12 will be to synchronize operation of the pump, and hence injection of blood into the coronary venous system, with the period of diastole. Thus, for example, control circuitry 14 will switch pump 12 on at the completion of systole (corresponding to the T-wave) and off at the offset of the QRS complex, in a manner similar to that employed in synchronized retroperfusion. Alternatively, control circuitry 14 may activate pump 12 only during systole.

As a yet further alternative, the duty cycle input into control circuitry 14 may require pump 12 to be continuously active for several seconds, alternating with several seconds of rest (e.g., 15 seconds on, followed by 4 seconds off). In this case, the limit values input to control circuitry 14, such as flow rate or pressure-related parameters, may be used to control operation of the pump. Thus, for example, pump 12 may be continuously on until a parameter related to the pressure or flow attains some predetermined value, after which the pump is shut off for several seconds.

It is expected that when implanted in the heart, apparatus 10 will provide short-term retrograde perfusion of the myocardium using the cardiac venous system, and will cause a redistribution of flow within the venous system so that a greater fraction of the deoxygenated blood exits via the lymphatic system and the Thebesian veins. While the venous system is not co-extensive with the coronary arteries (particularly with respect to the right ventricle), it is nevertheless expected that the method and apparatus of the present invention will provide short-term relief and preservation of ischemic myocardium in the majority of cases, since right ventricular infarcts are less common.

As described hereinabove, apparatus 10 may be implanted in a patient suffering from ischemic heart disease to reduce the load on the heart and preserve the myocardium from further infarction pending corrective surgery (i.e., either cardiac bypass surgery, heart replacement, or angioplasty). In addition, in accordance with the methods of the present invention, apparatus 10 may be left in position in the patient during a cardiac bypass operation or angioplasty procedure to preserve the myocardium. Upon completion of the corrective procedure, apparatus 10 then may be advantageously used to reduce the load on the left ventricle during revival of the heart and weaning of the patient from the cardiopulmonary bypass.

In addition to the foregoing uses, apparatus 10 may be advantageously used prior to corrective surgery in a diagnostic role. Specifically, regions of left ventricle dysfunction may be determined by comparing the distribution of nuclear isotopes, such as Technicium and Thallium, when the heart is at rest or stressed, to the distribution of isotopes observed after a period of retroperfusion via the coronary venous system. Such comparisons may yield important information with respect to, for example, how many bypass grafts are required and preferred locations for placement of such grafts, as described in the above-mentioned article to Udelson.

Referring now to FIG. 6, an alternative embodiment of the apparatus of the present invention is described. Apparatus 60 comprises conduit 80, conduit 90 and hydraulically-actuated pump 100. As illustrated in FIG. 6, inlet end 81 of conduit 80 is configured to be inserted via a femoral artery and through aorta A and aortic valve AV into left ventricle LV. Conduit 90 is configured to be inserted via a femoral vein and through inferior vena cava IVC and right atrium RA into the coronary sinus via the coronary ostium CO.

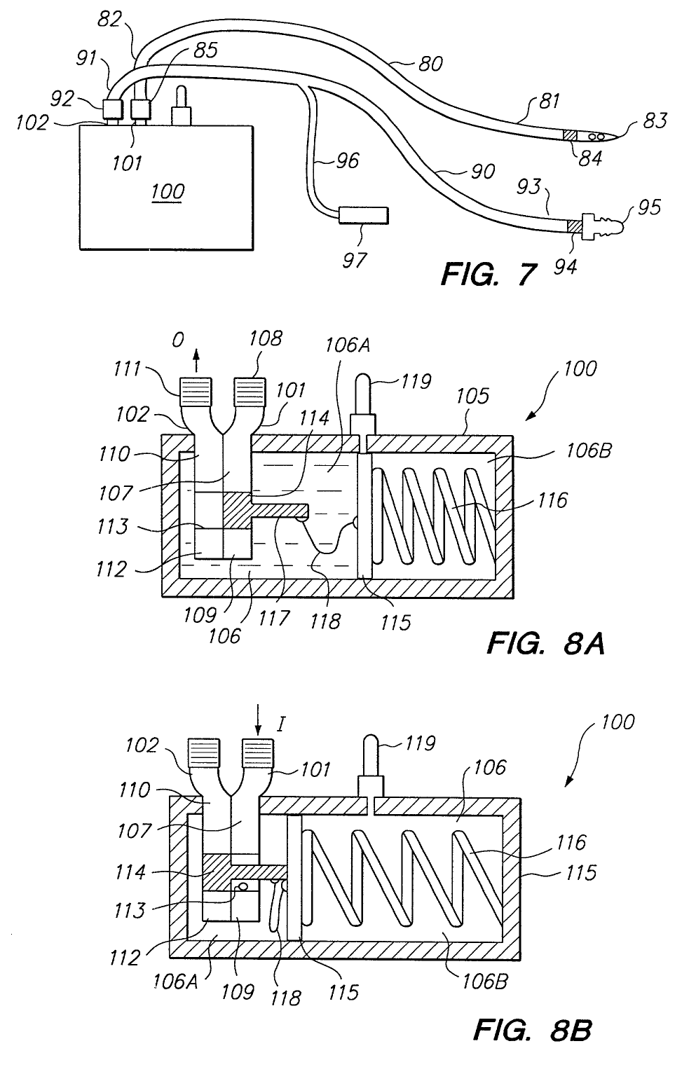

With respect to FIG. 7, conduit 80 is similar to conduit 20 described hereinabove, and includes inlet end 81, outlet end 82, tapered tip 83, radio-opaque marker band 84 and fitting 85. Conduit 90 is similar to conduit 30 described hereinabove, and includes inlet end 91 having fitting 92 and outlet end 93 having radio-opaque marker band 94 and plug 95 that engages the coronary sinus and partially or fully occludes the coronary ostium. Conduit 90 also may include branch 96 including fitting 97 to permit air to be removed from the fluid circuit, for example, by injecting saline solution.

Pump 100 includes inlet 101 that accepts fitting 85 of outlet end 82 of conduit 80, and an outlet 102 that accepts fitting 92 of inlet end 91 of conduit 90. Pump 100 preferably serves as an accumulator into which a volume of oxygenated blood is pumped by the left ventricle, and includes an hydraulically-actuated mechanism for periodically forcing the accumulated blood into the coronary sinus via conduit 90. Thus, hydraulic energy is transmitted to, and stored in, the mechanism as blood flows into the accumulator, and periodically released to pump blood from the accumulator into conduit 90.

Referring to FIGS. 8A and 8B, a first illustrative embodiment of hydraulically-actuated pump 100 constructed in accordance with the principles of the present invention is described. Pump 100 comprises housing 105 forming chamber 106. Inlet 101 comprises tube 107 having fitting 108 that engages fitting 85 of conduit 80, and outlet 109 that communicates with chamber 106. Outlet 102 comprises tube 110 having fitting 111 that engages fitting 92 of conduit 90, and inlet 112 that communicates with chamber 106. Tubes 107 and 110 are connected by manifold 113 in which valve 114 is reciprocated, as described hereinbelow.

Piston 115 is disposed within housing 105 in contact with spring 116. Piston 115 preferably forms a fluid tight seal that retains fluid in volume 106A of chamber 106, while preventing seepage of fluid into volume 106B containing spring 116. Valve 114 includes rod 117, which is coupled to the face of piston 115 by strand 118. Housing 105 optionally may include cartridge 119 which communicates with volume 106, and dispenses a metered amount of drug or tissue growth agent when chamber 106 is filled and volume 106B is compressed a predetermined degree.

Where the apparatus of FIG. 6 is used to provide retroperfusion during a beating-heart surgical procedure, such as a CABG procedure or angioplasty, housing 105 may be submerged in a cooling bath (not shown), or cartridge 119 may be used to dilute blood passing through chamber 106 with chilled saline. In this manner, a mild degree of hypothermia may be induced in the myocardium to further preserve ischemic regions.

Valve 114 is disposed in manifold 113 so that the valve block inlet 112 of tube 110 when blood is being accumulated in volume 106A of chamber 106, and blocks outlet 109 of tube 107 when piston 115 is ejecting the fluid from within chamber 106 into conduit 90. In FIG. 8A, pump 100 is shown in a state wherein blood (indicated by arrow O) previously accumulated in volume 106A of chamber 106 is being ejected by piston 115. In particular, valve 114 is shown blocking tube 107, and blood in volume 106A is ejected through outlet 102 into conduit 90 by the force exerted by spring 116.

As piston 115 ejects the blood from chamber 106 (e.g., by moving to the left in FIG. 8A), piston 115 contacts rod 117 and moves valve 114 so that it slides from a position blocking inlet 101 (in FIG. 8A) to a position blocking outlet 102 (see FIG. 8B). Once valve 114 closes tube 110 of outlet 102, blood (indicated by arrow I) is pumped into chamber 106A through conduit 80 and outlet 109 by the left ventricle. Blood thereby accumulates in volume 106A, causing spring 116 to become compressed. Cartridge 119, if provided, preferably is configured to inject a metered amount of a drug, e.g., an anti-clotting drug, such a heparin, or a tissue growth agent, such a VEG-F, into volume 106A. When volume 106A becomes full, strand 118 is pulled taut, and causes valve 114 to block outlet 109 of tube 107 and open inlet 112 of tube 110, thus causing valve 114 to return to the position shown in FIG. 8A.

Pump 100 serves as an accumulator to store blood injected into chamber 106 over the course of several heartbeats, and periodically and asynchronously injects the accumulated fluid into the coronary venous vasculature. Volume 106A of pump 100 preferably is from 10 to 100 ml of blood, and spring force 116 is selected to provide a flow rate, during outflow through conduit 90, of between 5-100 ml/sec. It is expected that pump 100 therefore will provide a mechanism to enhance perfusion and washout of metabolites from ischemic myocardium. Pump 100 may be initially filled with saline solution via fitting 97 and branch 96 to flush air out of the system.

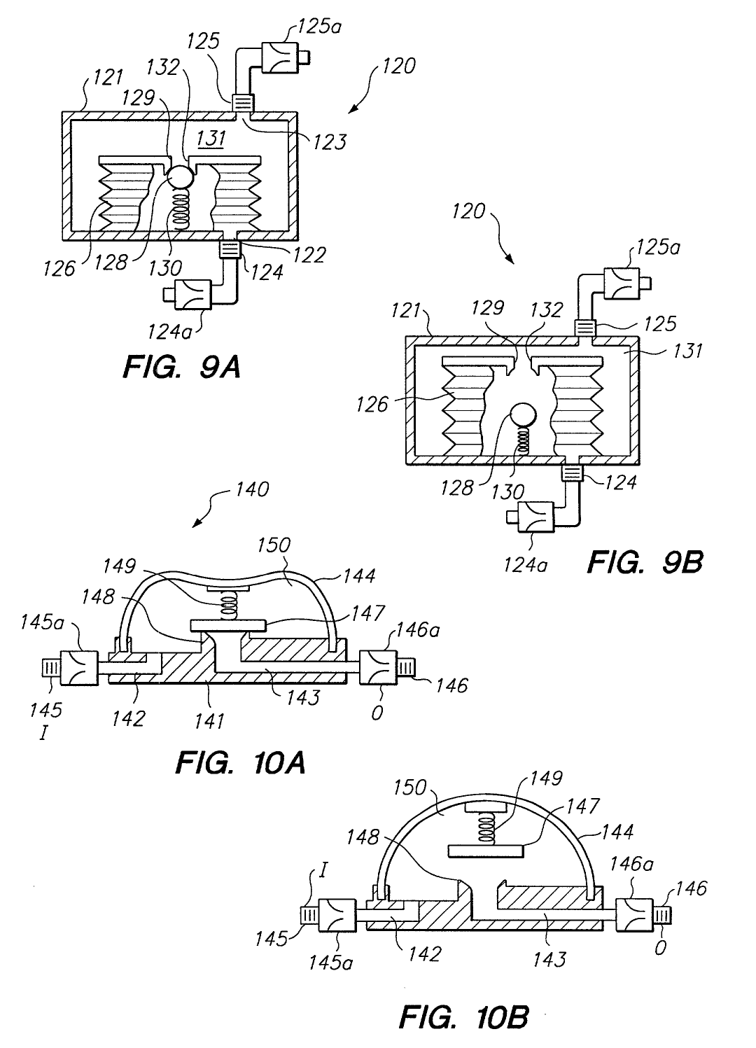

Referring now to FIGS. 9A and 9B, alternative pump 120 constructed in accordance with the principles of the present invention is described. Pump 120, which may be substituted for pump 100 of FIGS. 6 and 7, includes housing 121 having inlet 122 and outlet 123. Inlet 122 includes one-way valve 124a and fitting 124 that engages fitting 85 of conduit 80, while outlet 123 includes fitting 125 that engages fitting 92 of conduit 90 and one-way valve 125a. one-way valve 124a prevents blood injected into bellows 126 during systole from flowing in the reverse direction during diastole.

Inlet 122 opens into bellows 126 (shown partly cut-away), which is biased to maintain a collapsed position. Ball 128 sits in seat 129 and is biased away from seat 129 by spring 130. Housing 121 defines variable size volume 131 (depending upon the extension of bellows 126) that communicates with outlet 123. Bellows 126 includes opening 132 in seat 129 that permits volume 131 to communicate with the interior of the bellows when ball 128 is pulled free of seat 129.

Operation of pump 120 is as follows: during an inflow state, shown in FIG. 9A, blood accumulates within bellows 126, causing blood in volume 131 to be displaced through one-way valve 125a into conduit 90. Ball 128 remains seated in seat 129 against the bias force of spring 130, due to the pressure differential between the interior of bellows 126 and volume 131, which is proportional to that between the left ventricle and the coronary sinus. As bellows 126 fills with blood pumped from the left ventricle via conduit 80, the bellows expands.

At a predetermined degree of expansion of bellows 126, determined by the bias force of spring 130, the force applied by spring 130 overcomes the pressure differential that keeps ball 128 in seat 129. Ball 128 therefore is pulled way from seat 129, as shown in FIG. 9B, allowing bellows 126 to contract, and transferring the blood inside the bellows into volume 131. After bellows 126 contracts a predetermined amount, ball 128 again becomes seated in seat 129, and the above-described cycle of operation is repeated.

Referring to FIGS. 10A and 10B, a further alternative of an hydraulically-actuated pump constructed in accordance with the present invention is described. Pump 140, which also may be substituted for pump 100 of FIGS. 6 and 7, includes housing 141 having inlet 142, outlet 143 and dome 144. Inlet 142 includes one-way valve 145a and fitting 145 that engages fitting 85 of conduit 80, while outlet 143 includes optional one-way valve 146a and fitting 146 that engages fitting 92 of conduit 90. Dome 144 preferably comprises a compliant material, such as an elastomer, or a metal-alloy having a deflected position in the relaxed state, as shown in FIG. 10A.

Inlet 142 opens into volume 150 defined by dome 144 and an upper surface of housing 141. Poppet 147 is biased against seat 148 by spring 149. Poppet 147 sits atop seat 148, and blocks flow from volume 150 from exiting dome 144 via outlet 143. One-way valve 145a prevents blood injected into dome 144 from returning to the left ventricle during diastole.

Operation of pump 140 is as follows: during an inflow state, shown in FIG. 10A, spring 149 causes poppet 147 to remain seated in seat 148 until blood flowing into the dome through one-way valve 145a causes the dome to expand. As dome 144 fills with blood pumped from the left ventricle via conduit 80, dome 144 either expands radially outward (if a compliant material) or deflects outwardly, as depicted in FIG. 10B. At a predetermined degree of expansion or deflection of dome 144, sprint 149 pulls poppet 147 away from seat 148, as shown in FIG. 10B, allowing dome 144 to return to its unexpanded, or undeflected, state. When dome 144 contracts, blood accumulated within volume 150 is ejected through outlet 143, one-way valve 146a, and conduit 90 into the coronary venous vasculature. When dome 144 again contracts a predetermined amount, poppet 147 again contacts seat 148, and the above-described cycle of operation is repeated.

Accordingly, like the embodiments of FIGS. 8 and 9, pump 140 provides a hydraulically actuated device that accumulates blood from the left ventricle, thus reducing the load on the left ventricle, and asynchronously pumps that blood into the coronary venous vasculature to enhance perfusion. Also, like the embodiments of FIGS. 8 and 9, pump 140 requires no external power source, but instead stores hydraulic energy transmitted from the left ventricle over the course of several cardiac cycles in a mechanism that permits that energy to be periodically recovered to infuse blood into the coronary venous vasculature.

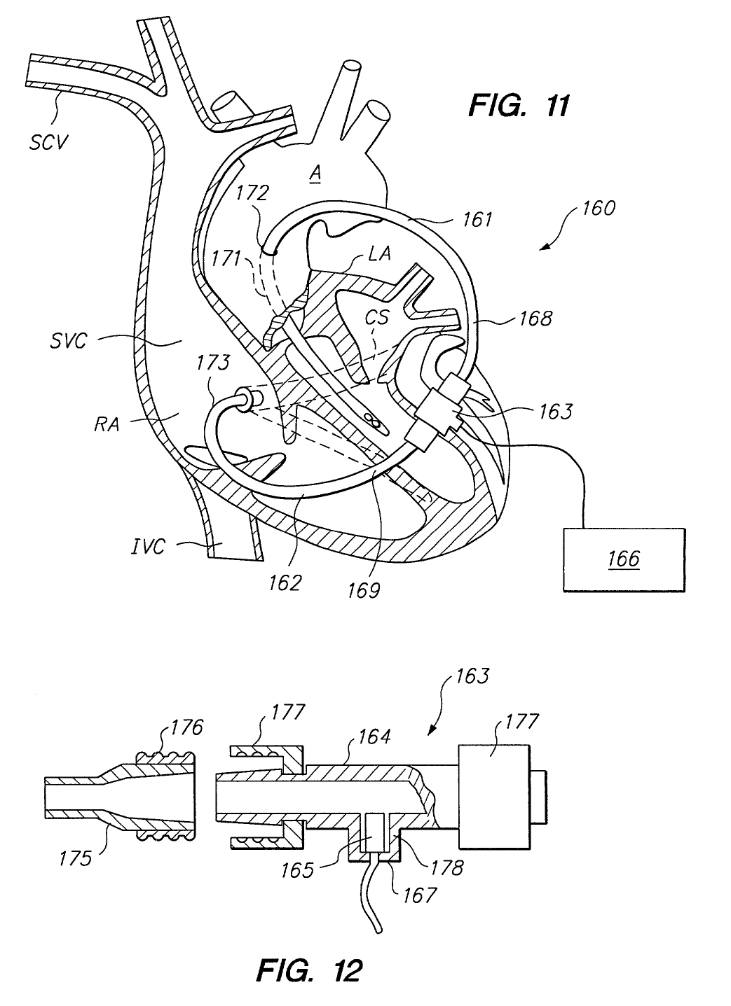

Referring now to FIGS. 11 and 12, another alternative embodiment of the apparatus and methods of the present invention are described. Apparatus 160 comprises inlet conduit 161, outlet conduit 162 and coupler 163. Coupler 163 may include housing 164 enclosing sensor 165. Sensor 165 is in turn coupled to monitoring system 166, which may be a previously known flow, pressure or other type of monitor, via port 167. Coupler 163 enables proximal end 168 of inlet conduit 161 to be coupled to proximal end 169 of outlet conduit 162. Alternatively, or in addition, coupler 163 may include additional ports 167 for monitoring other parameters, or for injecting drugs, bioactive agents, or cooled saline, as described above with respect to the embodiment of FIGS. 8A and 8B.

Still referring to FIG. 11, apparatus 160 may be installed using keyhole surgical or endoscopic techniques, so that distal end 171 of inlet conduit 161 enters aorta A through opening 172 formed through the wall of the aorta. Opening 172 may be closed around inlet conduit 161 using a purse string suture (not shown), as is per se known. Distal end 171 of inlet conduit 161 may be routed through the aortic valve and into the left ventricle (as shown in FIG. 11), or simply left in the aorta. Distal end 173 of outlet conduit 162 is disposed through the coronary ostium into the coronary sinus via an opening formed in the wall of the superior vena cava or right atrium, which also may be closed around the outlet conduit via a purse string suture (not shown).

Each of proximal ends 168 and 169 of inlet and outlet conduits 161 and 162, respectively, includes luer 175 having external ears or threads 176. As shown in FIG. 12, coupler 163 includes locking rings 177 that engage threads 176 and lock the conduits to the coupler. Preferably, sensor 165 is disposed in cavity 178 and port 167 to measure a flow-related parameter, such as flow rate or pressure, as described hereinabove. Alternatively, port 167 may be used to inject drugs, bioactive agents, or angiogenic growth factors or genes, or cooled saline.

In accordance with one aspect of the present invention, apparatus 160 may be implanted shortly before surgery for diagnostic purposes, as described hereinabove. Apparatus may then be left in position during a beating heart procedure, such as keyhole CABG or angioplasty, to perfuse and/or mildly cool (e.g., by 2-5° C.) the myocardium to preserve ischemic regions. In particular, if a CABG procedure is being performed, the distal end of a graft may first be anastomosed to the cardiac artery distal to the occluded region. Inlet catheter 161 may then be withdrawn through opening 172, and the proximal end of the graft anastomosed to opening 172 in aorta A, thus reducing the number of entry points into the aorta required to complete the bypass procedure.

While preferred illustrative embodiments of the invention are described above, it will be obvious to one skilled in the art that various changes and modifications may be made therein without departing from the invention and the appended claims are intended to cover all such changes and modifications which fall within the true spirit and scope of the invention.

What is claimed is:

1. A method of providing retrograde transvenous myocardial perfusion, the method comprising steps of:
providing a first conduit configured for insertion into a patient's left atrium, left ventricle or aorta and a second conduit configured for insertion into a portion of a patient's coronary venous vasculature;
inserting an inlet end of the first conduit into a patient's left atrium, left ventricle or aorta;
in inserting an outlet end of the second conduit into a patient's coronary venous vasculature; and
coupling an outlet end of the first conduit to an inlet end of the second conduit to enable a volume of blood to be removed from the left atrium, left ventricle or aorta via the first conduit and injected into the patient's coronary venous vasculature via the second conduit.

2. The method of claim 1 further comprising providing a chamber coupled between the outlet end of the first conduit and the inlet end of the second conduit, the chamber defining a volume that accumulates blood over several cardiac cycles before injecting the blood into the patient's coronary venous vasculature via the second conduit.

3. The method of claim 1 wherein the chamber further comprises a mechanism disposed within the chamber having a first state wherein the mechanism stores hydraulic energy transmitted by blood entering the chamber and a second state wherein the mechanism periodically releases the stored energy to pump blood into to the second conduit, the method further comprising:
storing hydraulic energy in the mechanism during inflow of blood into the chamber from the first conduit; and
releasing the hydraulic energy stored in the mechanism to inject blood into the patient's coronary venous vasculature via the second conduit.

4. The method of claim 1 further comprising:
coupling a motor-driven pump between the outlet of the first conduit and the inlet of the second conduit; and
controlling the motor-driven pump to limit a parameter related to a pressure attained within the coronary venous vasculature to a value less than a predetermined value.

5. The method of claim 4 further comprising steps of:
providing control circuitry; and
programming the control circuitry to control the pump with a predetermined duty cycle.

6. The method of claim 1 wherein inserting an outlet end of the second conduit into a patient's coronary venous vasculature comprises partially or fully occluding the coronary ostium.

7. The method of claim 6 wherein inserting the outlet end of the second conduit comprises transluminally inserting the second conduit via a rout including a femoral vein, inferior vena cava, and right atrium.

8. The method of claim 6 wherein inserting the outlet end of the second conduit comprises transluminally inserting the second conduit via a route including a superior vena cava and right atrium.

9. The method of claim 6 wherein inserting the outlet end of the second conduit comprises inserting the second conduit through a opening formed in a wall of the superior vena cava or right atrium.

10. The method of claim 1 wherein inserting an inlet end of the first conduit into a patient's left atrium or left ventricle further comprises forming a transseptal passageway and inserting the inlet end of the first conduit through the transseptal passageway.

11. The method of claim 1 wherein inserting an inlet end of the first conduit into a patient's left atrium, left ventricle or aorta comprises transluminally inserting the first conduit into the patient's left ventricle via a route including a femoral artery.

12. The method of claim 1 wherein inserting an inlet end of the first conduit into a patient's left atrium, left ventricle or aorta comprises inserting the inlet end through a opening formed in the wall of the aorta.

13. The method of claim 1 further comprising:
coupling a drug infusion device in fluid communication with the first and second conduits; and
infusing a predetermined amount of a therapeutic agent into the volume of blood prior to injecting the blood into the patient's coronary venous vasculature via the second conduit.

14. The method of claim 1 further comprising:
coupling a source of cooled saline in fluid communication with the first and second conduits; and
infusing a predetermined amount of a cooled saline into the volume of blood prior to injecting the blood into the patient's coronary venous vasculature via the second conduit to induce a mild state of hypothermia.

15. Apparatus for providing retrograde transvenous myocardial perfusion, the apparatus comprising:
a first conduit having an inlet end, an outlet end and a lumen extending between the inlet end and the outlet end, the inlet end configured to be inserted into a patient's left atrium, left ventricle or aorta;
a second conduit having an inlet end, an outlet end and a lumen extending between the inlet end and the outlet end, the outlet end configured to be inserted into a patient's coronary venous vasculature;
means for coupling the outlet end of the first conduit to the inlet end of the second conduit to enable a volume of blood to be removed from the left atrium, left ventricle or aorta via the first conduit and injected into the patient's coronary venous vasculature via the second conduit.

16. The apparatus of claim 15 further comprising:
a drug infusion device coupled in fluid communication to the first and second conduits, the drug infusion device infusing a predetermined amount of a therapeutic agent into the volume of blood injected into the patient's coronary venous vasculature via the second conduit.

17. The apparatus of claim 15 further comprising:
means for infusing a predetermined amount of a cooled saline into the volume of blood injected into the patient's coronary venous vasculature via the second conduit to induce a mild state of hypothermia.

18. The apparatus of claim 15 further comprising:
means coupled in fluid communication to the first and second conduits for monitoring a flow-related parameter for the volume of blood injected into the patient's coronary venous vasculature via the second conduit.

19. The apparatus of claim 15 further comprising:
a chamber coupled between the outlet end of the first conduit and the inlet end of the second conduit, the chamber having a volume sufficient to accumulate blood over several cardiac cycles.

20. The apparatus of claim 19 further comprising:
a drug infusion device coupled to the chamber, the drug infusion device infusing a predetermined amount of a therapeutic agent into blood accumulated in the chamber.

21. The apparatus of claim 15 further comprising a pump coupled between the outlet end of the first conduit and the inlet end of the second conduit.

22. The apparatus of claim 21 further comprising:
a chamber coupled between the outlet end of the first conduit and the inlet end of the second conduit, the chamber having a volume sufficient to accumulate blood over several cardiac cycles.

23. The apparatus of claim 22 further comprising:
a drug infusion device coupled to the chamber, the drug infusion device infusing a predetermined amount of a therapeutic agent into blood accumulated in the chamber.

24. The apparatus of claim 21 wherein the pump is motor-driven, the apparatus further comprising control circuitry for actuating the pump responsive to user selected input.

25. The apparatus of claim 24 wherein the control circuitry controls activation of the pump to limit a parameter related to a pressure attained within the coronary venous vasculature to a value less than a predetermined value.

26. The apparatus of claim 25 wherein the control circuitry is programmed by the user selected input to activate the pump with a predetermined duty cycle.

27. The apparatus of claim 22 wherein the pump comprises a mechanism having a first state wherein the mechanism stores hydraulic energy transmitted by blood entering the chamber and a second state wherein the mechanism periodically releases the stored energy to pump blood into the second conduit.

28. The apparatus of claim 27 further comprising:
a drug infusion device coupled to the chamber, the drug infusion device infusing a predetermined amount of a therapeutic agent into blood accumulated in the chamber.

29. The apparatus of claim 15 wherein the outlet end of the second conduit includes a plug that partially or fully occludes the coronary ostium.

30. The apparatus of claim 29 wherein the outlet end of the second conduit comprises a retention mechanism that engages an interior wall of a portion of the coronary venous vasculature.

31. The apparatus of claim 15 wherein the second conduit is further configured to be disposed in the patient's coronary venous vasculature via a route including a femoral vein and inferior vena cava.

32. The apparatus of claim 15 wherein the second conduit is further configured to be disposed in the patient's coronary venous vasculature via a route including a superior vena cava and right atrium.

33. The apparatus of claim 15 wherein the inlet end of the first conduit is configured to pass through a transseptal passageway formed between the patient's right and left atria.

34. The apparatus of claim 15 wherein the first conduit is configured to be transluminally disposed in the patient's left ventricle via a route including a femoral artery.

35. The apparatus of claim 15 wherein the first conduit is configured to be disposed in the patient's left ventricle or aorta via an opening formed through a wall of the aorta.

36. The apparatus of claim 15 wherein the inlet end of the first conduit comprises a portion defining a plurality of lateral openings that communicate with the lumen of the first conduit.

37. The apparatus of claim 15 further comprising a radio-opaque marker band disposed on one of the inlet end of the first conduit and the outlet end of the second conduit.

38. The apparatus of claim 37 wherein the outlet end of the second conduit further comprises a portion defining an opening to vent blood to the right atrium.

39. The apparatus of claim 15 wherein the inlet conduit further comprises a first luer disposed on a proximal end, the outlet conduit comprises a second luer disposed on a proximal end, and the means for coupling comprises a coupler that engages the first luer in fluid communication with the second luer.

40. The apparatus of claim 39 wherein the coupler further comprises means for monitoring a flow-related parameter for the volume of blood injected into the patient's coronary venous vasculature via the second conduit.
